(19) United States
(12) Patent Application Publication
(10) Pub. No.: US 2026/0308605 A1
(43) Pub. Date: Oct. 8, 2026

(54) PLANTS COMPRISING BROMOFORM PRODUCTION GENES

(71) Applicant: IMI TAMI INSTITUTE FOR RESEARCH & DEVELOPMENT LTD., Haifa (IL)

(72) Inventor: Michael Berson, Moshav Beit Hanan (IL)

(21) Appl. No.: 19/475,770

(22) PCT Filed: Apr. 16, 2024
(86) PCT No.: PCT/IL2024/050374

§ 371 Date: Oct. 16, 2025

Related U.S. Application Data
(60) Provisional application No. 63/460,095, filed on Apr. 18, 2023.

(51) Int. Cl. C12N 15/82; C12N 9/00; C12N 9/02; C12N 9/08; C12N 9/10; C12N 9/12

(52) U.S. Cl. CPC C12N 15/8243; C12N 9/0036; C12N 9/0065; C12N 9/1018; C12N 9/1029; C12N 9/1288; C12N 9/93; C12Y 106/03001; C12Y 201/03; C12Y 203/0118; C12Y 207/08007; C12Y 602/01

(57) ABSTRACT

The present invention provides a system of recombinant nucleic acid molecules including two or more genes encoding two or more enzymes involved in bromoform production for expression in plants, bromoform-producing plants or plant parts prepared by transforming a plant with the system, and methods for using the transformed plant or plant parts for feeding ruminant animals, in order to reduce methane emission caused by ruminants.

### FIELD OF THE INVENTION

The present disclosure is generally directed to transgenic plants. Specifically, the invention relates to transgenic plants for use in lowering greenhouse gas emissions in ruminant animals.

### BACKGROUND OF THE INVENTION

Agriculture has a key role in food production worldwide and it is a major component of the gross domestic product of several countries. Livestock production is essential for the generation of high-quality protein foods and the delivery of foods in regions where animal products are the main food source. Environmental impacts of livestock production have been examined for decades, but recently emission of methane from enteric fermentation has been targeted as a substantial greenhouse gas source.

Methane (CH₄) is recognized as the second most important greenhouse gas emitted by anthropogenic sources, and largely results from the microbial activity of methanogenic bacteria in the first stomach of ruminants (rumen). Ruminants are animals which have a multi-chamber stomach that relies on bacteria to break down plant-based food. In this digestive process, a fully grown cow can release up to 500 liters of methane into the atmosphere each day, contributing to approximately 17% of total global anthropogenic methane emissions.

Asparagopsis taxiformis, is a species of red algae. Researchers have demonstrated that feeding cows a diet containing one to two percent of this algae reduced their methane emissions by over 90%. As further shown, this effect was obtained without compromising other fermentation parameters (i.e. volatile fatty acid production), and with A. taxiformis organic matter inclusion rates of up to 5%. This effect was found to be due to inhibition of the fermentation by methanogens in the rumen by brominated organic hydrocarbons such as bromoform, present in A. taxiformis. However, bromoform-producing seaweeds such as A. taxiformis grow relatively slowly in deep seawater and therefore large scale production thereof is not cost-effective.

Several attempts have been made to clone vanadate-dependent haloperoxidases (VHPOs) from algae and bacteria into various organisms, however, this has been difficult due to low yield and toxicity issues.

Accordingly, there is still a need for an efficient system for feeding livestock while reducing methane emissions.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The invention relates to the preparation of transgenic pasture plants and fodder plants which carry bromoform-production genes from red or brown algae, in order to reduce methane emission caused by ruminants.

Advantageously, the genetically engineered bromoform-producing pasture plants can be seeded in grazing fields in order to be consumed by ruminant animals. Alternatively, foodstuffs may be prepared from the genetically engineered bromoform-producing plants and fed to ruminant animals.

In some embodiments, the present invention provides a system for gene expression in a plant, including one or more recombinant nucleic acid molecules which include one or more nucleotide sequences together including two or more genes, wherein: each gene is operably linked to a promoter for expression in the plant; and the two or more genes encode two or more enzymes involved in bromoform production and not encoded by the native plant.

In some embodiments, the two or more genes are included in a single nucleic acid molecule. In some embodiments, the two or more genes are included in two or more nucleic acid molecules.

In some embodiments, at least one of the two or more genes is from an organism selected from Asparagopsis taxiformis, Asparagopsis armata, Chondrus Crispus, Macrocystis pyrifera, Medicago sativa, and Medicago truncatula.

In some embodiments, at least two of the two or more genes are from at least one non-plant organism.

In some embodiments, the two or more enzymes include at least one haloperoxidase.

In some embodiments, the two or more enzymes include an NAD(P)H Oxidase (NOX).

In some embodiments, the two or more enzymes include at least one enzyme involved in fatty acid synthesis.

In some embodiments, the at least one enzyme involved in fatty acid synthesis is selected from Beta-ketoacyl-[acyl-carrier-protein]synthase III (FabH), phosphopantetheinyl transferase (sfp), acetyl-CoA carboxylase (ACCase), and malonyl CoA synthetase (MatB).

In some embodiments, the two or more enzymes include haloperoxidase, NOX, and at least one enzyme involved in fatty acid synthesis selected from FabH, sfp, ACCase, and MatB.

In some embodiments, the two or more enzymes include haloperoxidase, NOX, FabH, sfp, and ACCase.

In some embodiments, the two or more genes are selected from Mbb1, Mbb2, Mbb3, Mbb4, CcVHPO1, CcVHPO2, CcVHPO3, CcVHPO4, CcVHPO5, CcMbb2, Sfp, FaBH, ACCase, and MatB.

In some embodiments, the two or more genes include Mbb1, Mbb2, Mbb3, Mbb4, and at least one gene selected from FaBH, Sfp, ACCase, and MatB.

In some embodiments, the two or more genes include Mbb1, Mbb2, Mbb3, Mbb4, FaBH, Sfp, and ACCase.

In some embodiments, the haloperoxidase has at least 70, 75, 80, 85, 90, 95, or 99% sequence identity to SEQ ID NO: 1 (Mbb1), SEQ ID NO: 3 (Mbb3), or SEQ ID NO: 4 (Mbb4); the NOX has at least 70, 75, 80, 85, 90, 95, or 99% sequence identity to SEQ ID NO: 2 (Mbb2); the FabH has at least 70, 75, 80, 85, 90, 95, or 99% sequence identity to SEQ ID NO: 5; the sfp has at least 70, 75, 80, 85, 90, 95, or 99% sequence identity to SEQ ID NO: 6; and/or the ACCase has at least 70, 75, 80, 85, 90, 95, or 99% sequence identity to SEQ ID NO: 7.

In some embodiments, the two or more genes are operably linked to identical promoters. In some embodiments, the two or more genes are operably linked to different promoters. In some embodiments, at least one of the promoters is a constitutive promoter. In some embodiments, at least one of the promoters is an inducible promoter. In some embodiments, the inducible promoter is a tissue-specific promoter, activated in plant organs which develop late in the plant life cycle, such as leaves or flowers. In some embodiments, at least one of the two or more genes further encodes a chloroplast transit peptide for targeting the enzymes to the chloroplast.

In some embodiments, the plant is a pasture plant or a fodder plant. In some embodiments, the plant is a grass, a cereal, or a legume. In some embodiments, the plant is selected from signalgrass (Brachiaria), alfalfa (Medicago sativa, lucerne), ryegrass, tall fescue, cocksfoot, clover, oats, millet, kikuyu, chicory, corn, soy, and plantain.

In some embodiments, the present application provides a composition including the system disclosed herein.

In some embodiments, the present application provides a method for preparing a genetically engineered plant or plant part, the method including transforming a plant or a plant part with the system disclosed herein, or with the composition disclosed herein which includes the system disclosed herein.

In some embodiments, the present application provides a genetically engineered plant or plant part, including the system disclosed herein or prepared by the method for preparing a genetically engineered plant or plant part disclosed herein.

In some embodiments, the present application provides a genetically engineered plant or plant part, including one or more exogenous nucleotide sequences including two or more genes, wherein each gene is operably linked to a promoter for expression in the plant; and the two or more genes together encode two or more enzymes involved in bromoform production and not encoded by the native plant.

In some embodiments, the present application provides a genetically engineered plant cell, including the system disclosed herein or prepared by the method for preparing a genetically engineered plant or plant part disclosed herein.

In some embodiments, the present application provides a genetically engineered plant cell, including one or more exogenous nucleotide sequences including two or more genes, wherein each gene is operably linked to a promoter for expression in the plant; and the two or more genes together encode two or more enzymes involved in bromoform production and not encoded by the native plant.

In some embodiments, the plant is selected from signalgrass (Brachiaria), alfalfa (Medicago sativa, lucerne), ryegrass, tall fescue, cocksfoot, clover, oats, millet, kikuyu, chicory, corn, soy, and plantain.

In some embodiments, the plant part is selected from a seed, a cell, a leaf, a stem, a root, a tuber, a cutting, a flower, a bark, a fruit, a bulb, a trichome, a rhizome.

In some embodiments, the present application provides a composition including the genetically engineered plant or plant part disclosed herein mixed together with a plant or plant part not capable of producing bromoform, wherein the composition includes up to about 10% genetically engineered plant or plant part.

In some embodiments, the present application provides a method for reducing methane gas emission by a ruminant animal, the method including:
producing a genetically engineered seed of a pasture plant, the seed including two or more genes involved in bromoform production;
seeding the genetically engineered seed in a field where a ruminant animal grazes; and
growing a bromoform-producing pasture plant from the seed,
thereby causing the bromoform-producing pasture plant to be consumed by the ruminant animal.

In some embodiments, the genetically engineered seed is produced by the method for preparing a genetically engineered plant or plant part disclosed herein.

In some embodiments, the ruminant animal is a cow.

In some embodiments, the present application provides a method for preparing a foodstuff for reducing methane gas emission by a ruminant animal, the method including:
producing a genetically engineered seed of a fodder plant, the seed including two or more genes involved in bromoform production;
seeding the genetically engineered seed;
growing a bromoform-producing fodder plant from the seed; and
preparing a foodstuff containing the bromoform-producing fodder plant or a part thereof.

In some embodiments, the genetically engineered seed is produced by the method for preparing a genetically engineered plant or plant part disclosed herein.

In some embodiments, the ruminant animal is a cow.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

FIGS. 1A-1C present an RT-PCR analysis of transformed tobacco plants using specific primers to identify the integration and transcription of 7 or 4 bromoform-producing genes per transgenic plant (primers used are for the genes encoding resistance to kanamycin, spectinomycin and Basta used for the selection, see Table 2). Plants 1-3 show transcription in planta of kanamycin (FIG. 1A), spectinomycin (FIG. 1B), and Basta (FIG. 1C) resistance genes, that provide evidence for expression of all 7 genes: mbb1-4, sfp, fabH and ACCase (from plasmids 3-5), involved in bromoform production. Plants 4-6 show transcription in planta of kanamycin (FIG. 1A) and spectinomycin (FIG. 1B) resistance genes (but not the Bar (Basta resistance) gene (FIG. 1C)), that provide evidence for expression of 4 genes: mbb1-4 from plasmids 1+2. MW—molecular weight marker, wt—wild type tobacco plants, NTC—no template control.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The problem of methane emissions by ruminant animals has raised much attention and various solutions have been developed. Many of the solutions were based on the fact that red seaweed Asparagopsis taxiformis produces bromoform, which interferes with methane production in the stomach of the cow. For example, one approach involved feeding cows with Asparagopsis taxiformis, while another included transferring the genes encoding A. taxiformis haloperoxidase, the key enzyme participating in bromoform production, into the genome of various organisms, such as yeast and bacteria, in order to produce feedstuff including these bromoform-producing organisms. Each of the approaches had some shortcomings, including costs, toxicity of the bromoform, and low yield.

The present invention presents a novel approach to the problem, which involves transferring a combination of bromoform-production genes from A. taxiformis and additional organisms into plants, thereby allowing them to be seeded in grazing fields and be consumed by cows. One of the advantages of this method is that the method of providing the bromoform by grazing rather than by feeding may allow for a lower yield of the bromoform to be sufficient for achieving the methane-inhibiting effect.

### A System for Generating Bromoform Producing Plants

Accordingly, in some embodiments, there is provided a system for gene expression in a plant including one or more recombinant nucleic acid molecules which include one or more nucleotide sequences together including two or more genes, wherein: each gene is operably linked to a promoter for expression in the plant; and the two or more genes together encode two or more enzymes involved in bromoform production and not encoded by the native plant.

The phrase “one or more nucleotide sequences together including two or more genes” is intended to clarify that the total number of genes included in the system is two or more (at least two), and that this total number of genes may be included in a single nucleotide sequence or in two or more nucleotide sequences. In the same way it is clarified that the total of two or more genes may be included on one recombinant nucleic acid molecule, or on two or more recombinant nucleic acid molecules. Each nucleic acid molecule and each nucleotide sequence may include one gene, more than one gene, a part of a gene, no genes or parts thereof, or any combination of genes and/or parts thereof. In total, the system of recombinant nucleic acid molecules includes at least two genes involved in bromoform production, and encodes at least two enzymes involved in bromoform production.

In some embodiments, the two or more genes are included in a single nucleic acid molecule. In some embodiments, the two or more genes are included in two or more nucleic acid molecules.

The term “native plant” means the original plant, before adding the system of the invention. In some embodiments, “native plant” is a plant as found in nature, not including any genetic modifications.

The term “operably linked”, as used herein, means that the respective elements are arranged such that there is a functional relationship between them. For example, referring to a gene that is operably linked to a promoter means that the position and orientation of the gene and the promoter are appropriate such that the promoter is driving expression of the gene. It is appreciated that one promoter may be operably linked to more than one gene. It is also appreciated that a promoter may be operably linked to the gene via a trans-activating factor, such that the promoter drives expression of the trans-activating factor, which binds to a regulatory element which induces expression of the gene.

In some embodiments, more than one gene are operably linked to a single promoter. In some embodiments, each gene is operably linked to a separate promoter.

Bromoform (CHBr₃) is a brominated organic solvent, colorless liquid at room temperature, with a high refractive index, very high density, and sweet odor, similar to that of chloroform. Bromoform is known as an inhibitor of methanogenesis and is a common component of seaweed. The mechanism of inhibition is hypothesized to be by reducing the efficiency of the cobamide-dependent methyl transferase by interfering with vitamin B12 binding, which is a crucial step of methanogenesis in the rumen.

Several organisms are known as bromoform producers, including, but not limited to, the algae species Asparagopsis taxiformis (red sea plume or limu kohu, formerly A. sanfordiana), Asparagopsis armata, Chondrus crispus (Irish moss or carrageen moss), and Macrocystis pyrifera (giant kelp).

A. taxiformis is a species of red algae with a wide distribution in tropical to warm temperate waters. Researchers have demonstrated that feeding ruminants a diet containing as little as 0.2% A. taxiformis seaweed reduced their methane emissions by nearly 99%. Additional types of seaweed were tested, many were able to reduce methane emissions by over 90%. The active ingredient in the seaweed was found to be bromoform. A. armata is also a species of red sea algae which has been shown to be able to reduce methane production in dairy cows. C. crispus is a species of red algae also used as a source of the thickener carrageenan. Macrocystis pyrifera is a brown macroalga.

In some embodiments, the two or more genes are three or more, four or more, five or more, six or more, or seven genes.

In some embodiments, at least one of the two or more genes is from an organism selected from Asparagopsis taxiformis, Asparagopsis armata, Chondrus Crispus, Macrocystis pyrifera, Medicago sativa, and Medicago truncatula.

In some embodiments, at least two of the two or more genes are from at least one non-plant organism. In some embodiments, the at least one non-plant organism is an alga. In some embodiments, the alga is a red or brown alga. In some embodiments, the alga is a red alga. In some embodiments, the alga is selected from Asparagopsis taxiformis, Asparagopsis armata, Chondrus Crispus, and Macrocystis pyrifera. In some embodiments, the alga is Asparagopsis taxiformis.

Several enzymes have been identified as being involved in bromoform production. These enzymes include haloperoxidases (such as vanadate-dependent haloperoxidase, or VHPO), and NAD(P)H oxidase (NOX). Haloperoxidases catalyze the conversion of halide anions to hypohalous acid with hydrogen peroxide acting as the oxidant (Br⁻+H₂O₂=>HOBr+OH⁻). Hypohalous acid delivers electrophilic helenium ion which halogenates hydrocarbon substrates to furnish natural products such as bromoform (3HOBr+CH=>CHBr₃+3OH⁻). VHPOs use vanadium as a cofactor. The need for hydrogen peroxide in the peroxidase reaction links this pathway to enzymatic production of reactive oxygen species (ROS) by NOXs. NOXs are membrane bound enzymes that catalyze the transfer of electrons from the electron donor, NAD(P)H, via flavin and heme cofactors to molecular oxygen to generate ROS such as hydrogen peroxide and superoxide anion (dismutated to hydrogen peroxide).

Accordingly, in some embodiments, the two or more enzymes include at least one haloperoxidase. In some embodiments, the two or more enzymes include a NOX. In some embodiments, the two or more enzymes include at least one haloperoxidase and a NOX.

Additionally, as shown by Thapa 2020 (ACS Chem Biol. 15(6): 1662-1670), fatty acid biosynthesis intermediates, such as malonyl CoA, can provide substrates for the VHPO enzymes, and are therefore also considered to be involved in bromoform production. The enzymes participating in the relevant fatty acid synthesis reactions are encoded by the genes FabH (Beta-ketoacyl-[acyl-carrier-protein]synthase III), MatB (malonyl CoA synthetase), and Sfp (phosphopantetheinyl transferase). Briefly, malonate and CoA-SH are condensed by MatB to malonyl-coenzyme A. Sfp modifies ACP (acyl carrier protein) to malonyl-S-ACP by using the malonyl-coenzyme A. After incubation with acetyl-CoA and FabH, it is converted to acetoacetyl-S-ACP. The acetoacetyl-S-ACP can be incubated with a haloperoxidase to produce bromoform.

Malonyl CoA is more often produced from acetyl CoA by acetyl-CoA carboxylase (ACCase), instead of by malonyl CoA synthetase. ACCase is a biotin-dependent carboxylase that catalyzes the irreversible carboxylation of acetyl-CoA to produce malonyl-CoA through its two catalytic activities, biotin carboxylase and carboxyltransferase. The most important function of ACCase is to provide the malonyl-CoA substrate for the biosynthesis of fatty acids.

In some embodiments, the two or more enzymes are three or more, four or more, five or more, six or more, or seven enzymes.

Accordingly, in some embodiments, the two or more enzymes include at least one enzyme involved in fatty acid synthesis. In some embodiments, the at least one enzyme involved in fatty acid synthesis is selected from Sfp, FabH, ACCase, and MatB.

In some embodiments, the two or more enzymes include at least one haloperoxidase and at least one enzyme involved in fatty acid synthesis selected from FabH, sfp, ACCase, and MatB.

In some embodiments, the two or more enzymes include at least one haloperoxidase, NOX, and at least one enzyme involved in fatty acid synthesis selected from FabH, sfp, ACCase, and MatB.

In some embodiments, the two or more enzymes include at least one haloperoxidase, NOX, and FabH. In some embodiments, the two or more enzymes include at least one haloperoxidase, NOX, and sfp. In some embodiments, the two or more enzymes include at least one haloperoxidase, NOX, and ACCase.

In some embodiments, the two or more enzymes include at least one haloperoxidase, NOX, FabH, and sfp. In some embodiments, the two or more enzymes include at least one haloperoxidase, NOX, FabH, and ACCase. In some embodiments, the two or more enzymes include at least one haloperoxidase, NOX, sfp, and ACCase.

In some embodiments, the two or more enzymes include at least one haloperoxidase, FabH, sfp, and ACCase. In some embodiments, the two or more enzymes include at least one haloperoxidase, NOX, FabH, sfp, and ACCase.

In some embodiments, the at least one haloperoxidase is selected from Mbb1, Mbb3, and Mbb4, and combinations thereof. In some embodiments, the at least one haloperoxidase includes Mbb1, Mbb3, and Mbb4. In some embodiments, the NOX is Mbb2.

In some embodiments, the Mbb1 has at least 70, 75, 80, 85, 90, 95, or 99% sequence identity to SEQ ID NO: 1. In some embodiments, the Mbb1 has a sequence according to SEQ ID NO: 1.

In some embodiments, the Mbb2 has at least 70, 75, 80, 85, 90, 95, or 99% sequence identity to SEQ ID NO: 2. In some embodiments, the Mbb2 has a sequence according to SEQ ID NO: 2.

In some embodiments, the Mbb3 has at least 70, 75, 80, 85, 90, 95, or 99% sequence identity to SEQ ID NO: 3. In some embodiments, the Mbb3 has a sequence according to SEQ ID NO: 3.

In some embodiments, the Mbb4 has at least 70, 75, 80, 85, 90, 95, or 99% sequence identity to SEQ ID NO: 4. In some embodiments, the Mbb4 has a sequence according to SEQ ID NO: 4.

In some embodiments, the FabH has at least 70, 75, 80, 85, 90, 95, or 99% sequence identity to SEQ ID NO: 5. In some embodiments, the FabH has a sequence according to SEQ ID NO: 5.

In some embodiments, the sfp has at least 70, 75, 80, 85, 90, 95, or 99% sequence identity to SEQ ID NO: 6. In some embodiments, the sfp has a sequence according to SEQ ID NO: 6.

In some embodiments, the ACCase has at least 70, 75, 80, 85, 90, 95, or 99% sequence identity to SEQ ID NO: 7. In some embodiments, the ACCase has a sequence according to SEQ ID NO: 7.

In A. taxiformis, the relevant genes were identified and localized to a single locus termed the marine bromoform biosynthesis locus (GenBank accession No. MN966723 and MN893468). The genes identified in the Mbb locus include both VHPOs—Mbb1, Mbb3, and Mbb4, as well as a NOX—Mbb2. It is noted that Mbb2 is not the only NOX identified in A. taxiformis. Corresponding genes were also identified in the algae species Chondrus crispus and include the VHPO genes CcVHPO1-5 and the NOX gene CcMbb2 (Thapa et al., 2020, ACS Chem Biol. 15(6): 1662-1670). The above-identified VHPOs of A. taxiformis and of C. crispus are specific to bromine.

Accordingly, in some embodiments, the two or more genes are selected from Mbb1, Mbb2, Mbb3, Mbb4, CcVHPO1, CcVHPO2, CcVHPO3, CcVHPO4, CcVHPO5, CcMbb2, Sfp, FaBH, ACCase, and MatB.

Mbb1-4 were successfully expressed in E. coli, however, attempts to express Mbb2 in bacteria and yeast was unsuccessful and CcMbb2 was expressed in yeast instead. In addition, it is noted that Mbb3 was found not to produce bromoform and exhibited low expression level, and that CcVHPO4 and CcVHPO5 were not tested (Thapa et al. 2020, ACS Chem Biol. 15(6): 1662-1670).

In some embodiments, the two or more genes include at least mbb2 or CcMbb2.

It is further noted that some of the genes, especially the NOX and the fatty acid synthesis enzymes may be endogenously present in the plant, and therefore sometimes providing only haloperoxidases, or any other partial list of the above genes, may be sufficient.

In some embodiments, the two or more genes include the genes Mbb1 and Mbb2.

In some embodiments, the two or more genes include the genes Mbb3 and Mbb4.

In some embodiments, the two or more genes include the genes Mbb1, Mbb2, Mbb3, and Mbb4.

In some embodiments, the two or more genes include the genes Mbb1, Mbb3, Mbb4, and at least one gene selected from sfp, FabH, and ACCase. In some embodiments, the two or more genes include the genes Mbb1, Mbb2, Mbb3, Mbb4, and at least one gene selected from sfp, FabH, and ACCase.

In some embodiments, the two or more genes include Mbb1, Mbb2, Mbb3, Mbb4, and sfp. In some embodiments, the two or more genes include Mbb1, Mbb2, Mbb3, Mbb4, and FabH. In some embodiments, the two or more genes include Mbb1, Mbb2, Mbb3, Mbb4, and ACCase.

In some embodiments, the two or more genes include Mbb1, Mbb2, Mbb3, Mbb4, sfp, and FabH In some embodiments, the two or more genes include Mbb1, Mbb2, Mbb3, Mbb4, sfp, and ACCase. In some embodiments, the two or more genes include Mbb1, Mbb2, Mbb3, Mbb4, FabH, and ACCase.

In some embodiments, the two or more genes include Mbb1, Mbb3, Mbb4, sfp, FabH, and ACCase. In some embodiments, the two or more genes include Mbb1, Mbb2, Mbb3, Mbb4, sfp, FabH, and ACCase.

In some embodiments, the Mbb1, Mbb2, Mbb3, and/or Mbb4 genes are from Asparagopsis taxiformis or encode the same enzyme sequence encoded by the Asparagopsis taxiformis Mbb1, Mbb2, Mbb3, and/or Mbb4 genes, respectively. In some embodiments, the sfp gene is from Medicago truncatula or encodes the same enzyme sequence encoded by the Medicago truncatula sfp gene. In some embodiments, the FabH gene is from Chondrus crispus or encodes the same enzyme sequence encoded by the Chondrus crispus FabH gene. In some embodiments, the ACCase gene is from Medicago sativa or encodes the same enzyme sequence encoded by the Medicago sativa ACCase gene. In some embodiments, at least one of the two or more genes is an ortholog from a different species of a gene indicated above.

It is appreciated that any one of the genes of the invention may encode the same enzyme sequence encoded by a reference gene (e.g., the gene of the invention encodes the same enzyme sequence encoded by the Chondrus crispus FabH gene), but have a coding sequence different, or altered, compared to the respective gene. In some embodiments, the coding sequence is altered by using codon optimization. In some embodiments, the coding sequence is altered by using a codon usage scheme of a different organism. In some embodiments, the coding sequence is altered by using a codon usage scheme of Medicago sativa.

In some embodiments, the Mbb1 gene has at least 70, 75, 80, 85, 90, 95, or 99% sequence identity to a sequence encoding SEQ ID NO: 1.

In some embodiments, the Mbb2 gene has at least 70, 75, 80, 85, 90, 95, or 99% sequence identity to a sequence encoding SEQ ID NO: 2.

In some embodiments, the Mbb3 gene has at least 70, 75, 80, 85, 90, 95, or 99% sequence identity to a sequence encoding SEQ ID NO: 3.

In some embodiments, the Mbb4 gene has at least 70, 75, 80, 85, 90, 95, or 99% sequence identity to a sequence encoding SEQ ID NO: 4.

In some embodiments, the FabH gene has at least 70, 75, 80, 85, 90, 95, or 99% sequence identity to a sequence encoding SEQ ID NO: 5.

In some embodiments, the sfp gene has at least 70, 75, 80, 85, 90, 95, or 99% sequence identity to a sequence encoding SEQ ID NO: 6.

In some embodiments, the ACCase gene has at least 70, 75, 80, 85, 90, 95, or 99% sequence identity to a sequence encoding SEQ ID NO: 7.

In some embodiments, the two or more genes are all operably linked to identical promoters.

In some embodiments, at least two of the two or more genes are operably linked to promoters which are different from one another. In some embodiments, each one of the two or more genes is operably linked to a separate promoter. In some embodiments, more than one gene is operably linked to a single promoter.

One of the problems with bromoform production is that bromoform itself may be hazardous to the ozone layer. Accordingly, an advantage of the present invention is that it allows controlling the amount of bromoform produced in various ways, such as, by controlling expression of the bromoform production genes by limiting them to specific stages of development, specific states, or specific tissues. This may be achieved, for example, by using an inducible promoter, which limits expression of these genes to a specific time or place.

The promoter may be any promoter suitable for expression in plants. In some embodiments, the promoter is a constitutive promoter. In some embodiments, the promoter is an inducible promoter. In some embodiments, at least one of the genes is operably linked to an inducible promoter.

Nonlimiting examples for a constitutive promoter include, for example, the cauliflower mosaic virus 35S promoter, the Agrobacterium NOS promoter, and a promoter of a house-keeping genes such as actin (e.g. act8 promoter) or ubiquitin (ubq10 promoter), etc.

Nonlimiting examples for a tissue specific promoter include, e.g., the leaf-specific promoter of sedoheptulose-1, 7-bisphosphatase (SBPase, e.g. from B. distachyon), or the fructose-1, 6-bisphosphate aldolase (FBPA) promoter highly expressed in leaves.

The term “constitutive promoter” relates to a promoter which is always in an “on” state, i.e., driving transcription of a gene. It is generally not affected by regulatory factors, such as temporal factors, or tissue-specific factors. Examples for a constitutive promoter are promoters of housekeeping genes.

The term “inducible promoter” or “regulated promoter” relates to a promoter which is, as a default, in an “off” state, i.e., not driving transcription, and is turned on in response to specific regulatory factors, e.g., tissue-specific or developmental-specific factors. Nonlimiting examples for an inducible promoter include a tissue-specific promoter, and a developmentally regulated promoter.

In order to keep bromoform production to a minimum, thereby reducing excess bromoform which is harmful both to the plant itself and to the environment, an inducible promoter may be used, such that the relevant genes are only expressed when they are needed.

For example, according to some embodiments, the bromoform is only needed when the animal grazes. Accordingly, the expression of at least some of the bromoform production genes may be activated only when the plant reaches the appropriate stage of development and is ready to be eaten. Alternatively, bromoform production may be activated only when cows are grazing, turned on, e.g., by the stress response invoked by the damage done to the plant by the cow's bite.

Therefore, in some embodiments, the inducible promoter is a developmentally regulated promoter, induced by factors which are induced at late stages of development of the plant. In some embodiments, the inducible promoter is a tissue-specific promoter, activated in specific tissues which develop at a later stage in the plant life cycle, such as leaves or flowers.

In some embodiments, the inducible promoter is induced by factors which are activated due to being consumed by a ruminant animal, such as regulatory factors induced by stress, e.g., jasmonic acid. In some embodiments, the inducible promoter is a jasmonic acid promoter or a jasmonic acid-responsive promoter.

One of the problems of gene targeting in plants is suppression of the foreign genes by the plant defense systems. One approach which may help overcome this problem is targeting to an organelle in the plant cell, e.g., a plastid, such as the chloroplast. Targeting to chloroplast may have a two-fold advantage: the first is sequestration of the foreign genes, possibly protecting them from being silenced by the host plant; and the second is that fatty acid synthesis takes place in the chloroplast and can provide precursors for the haloperoxidase.

Accordingly, in some embodiments, at least one of the two or more genes further encodes a plastid targeting motif or a chloroplast targeting motif, such as a chloroplast transit peptide. In some embodiments, the two or more genes all further encode chloroplast transit peptides.

A chloroplast transit peptide is a short peptide that directs the transport of a protein to the chloroplast. The transit peptide is usually cleaved upon import. Chimeric chloroplast transit peptides are disclosed in WO2012161982, which is incorporated herein by reference.

Another approach is using the P19 viral RNA silencing suppressor, which may prevent the silencing of the foreign genes.

Accordingly, in some embodiments, the system further includes a sequence encoding the P19 viral RNA silencing suppressor gene.

It is appreciated that the recombinant nucleic acid molecules of the invention may further include additional elements, such as transcription terminators, regulatory elements, selective markers, etc., as needed for transformation and expression in plants. Non-limiting examples for selective markers include kanamycin resistance, spectinomycin resistance, and/or glufosinate or phosphinothricin herbicide resistance (E.g. the brand Basta).

In some embodiments, the one or more recombinant nucleic acid molecules are included in at least one vector suitable for transformation into plants, and/or expression in plants. In some embodiments, the vector is a binary vector. In some embodiments, the binary vector system is a Ti plasmid-based system in which a gene of interest is cloned into the T-DNA region of the plasmid, and transformation is conducted into Agrobacterium containing a a vir helper plasmid. In some embodiments, the vector is based on Ti plasmid. In some embodiments the vector is a pAB binary vector. In some embodiments the vector is a pPA binary vector.

The recombinant nucleic acid molecules of the invention may be prepared by any suitable genetic engineering method, including, for example, plasmid cloning, DNA synthesis, restriction digestion and ligation, T-DNA manipulation, clustered regularly interspaced short palindromic repeats (CRISPR) technology, polymerase chain reaction (PCR), etc.

In some embodiments, there is provided a composition including the system disclosed herein. In some embodiments, the composition further includes reagents suitable for transforming plants. Suitable reagents are well known in the art and also disclosed in the Examples.

In some embodiments, there is provided a composition including the system of the invention for use in transforming a plant. In some embodiments, there is provided a use of a composition including the system of the invention for transforming a plant.

### Methods of Preparing Bromoform-Producing Plants

In some embodiments, there is provided a method for preparing a genetically engineered plant or plant part, the method including transforming a plant or a plant part with the system disclosed herein or with the composition disclosed above.

Definitions and embodiments mentioned above and which may be relevant to the method of preparation embodiments also apply here, and vice versa. Some particularly relevant embodiments may be pointed out or explicitly repeated.

Transforming plants with the nucleic acids of the invention may be conducted by any suitable method known in the art. Nonlimiting examples for methods include transformation mediated by Agrobacterium, and/or transformation by the floral dip method (e.g., as described in WO 2018/178975).

In some embodiments, the method includes transforming the system of the invention into plant leaves. In some embodiments, the method includes transforming the system of the invention into plant seeds.

In some embodiments, the system of the invention includes more than one (at least two) recombinant nucleic acid molecule, and at least two of the recombinant nucleic acid molecules are transformed together into the plant. In some embodiments, the system of the invention includes more than one recombinant nucleic acid molecule, and at least two of the recombinant nucleic acid molecules are transformed separately into the plant. In some embodiments, the system of the invention includes more than one recombinant nucleic acid molecule, and all of the recombinant nucleic acid molecules are transformed together (co-transformed) into the plant.

It is appreciated that the genes of the invention may be inserted together or separately into the plant by any suitable method including using any genome editing system or method including systems using engineered nucleases selected from the group consisting of: meganucleases, zinc finger nucleases (ZFNs), transcription activator-like effector-based nucleases (TALEN), clustered regularly interspaced short palindromic repeats (CRISPR) system and any combination thereof.

In some embodiments, the method further includes a step of growing a plant from the plant part transformed by the system of the invention. In some embodiments, the method further includes a step of obtaining a plant part from the grown plant.

For example, the leaves of a plant may be transformed with the system of the invention, and a plant is grown from the transformed leaves. Seeds may then be obtained from the grown transformed plant in order to generate additional transformed plants.

In some embodiments, the method further includes a step of selecting for transformed plants. Selecting may be conducted by any suitable method known in the art, such as including a gene or genes encoding selectable markers, such as kanamycin resistance, spectinomycin resistance, and/or glufosinate or phosphinothricin herbicide resistance (E.g. the brand Basta), in the vectors transformed into the plant, and growing the transformants in the presence of the appropriate selection.

### Genetically Engineered Bromoform-Producing Plants and Plant Parts

In some embodiments, there is provided a genetically engineered plant or plant part including the system disclosed herein or prepared by the method for preparing a genetically engineered plant or plant part disclosed herein.

In some embodiments, there is provided a genetically engineered plant or plant part including one or more exogenous nucleotide sequences including two or more genes, wherein: each gene is operably linked to a promoter for expression in the plant; and the two or more genes together encode two or more enzymes involved in bromoform production and not encoded by the native plant.

In some embodiments, the present application provides a genetically engineered plant cell, including the system disclosed herein or prepared by the method for preparing a genetically engineered plant or plant part disclosed herein.

In some embodiments, the present application provides a genetically engineered plant cell, including one or more exogenous nucleotide sequences including two or more genes, wherein each gene is operably linked to a promoter for expression in the plant; and the two or more genes together encode two or more enzymes involved in bromoform production and not encoded by the native plant.

It is appreciated that the genetically engineered plants or plant parts disclosed herein are intended to be bromoform-producing, or to be capable of developing into bromoform-producing plants or plant parts. However, since at least some of the genes involved in bromoform production may be tissue-specific or developmentally-regulated, production of bromoform may be limited to certain tissues or plant parts. Accordingly, the term “plant part”, as used herein, encompasses a plant part which produces bromoform after being transformed with the constructs used (and provided with a bromine source), or a plant part which may not produce bromoform after being transformed, but may develop into a bromoform-producing plant or plant part. Examples for the latter may include a cell or a seed.

The terms “genetically engineered” and “transgenic” are used herein interchangeably and relate to plants or plant parts which include exogenous nucleotide sequences encoding bromoform-producing genes, as described herein.

The term “exogenous nucleotide sequence”, as used herein, means a nucleotide sequence which is not part of the native plant.

In some embodiments, there is provided a genetically engineered plant or plant part including two or more genes, wherein: each gene is operably linked to a promoter for expression in the plant; and the two or more genes together encode two or more enzymes involved in bromoform production.

Definitions and embodiments mentioned above and which may be relevant to the genetically engineered plant embodiments also apply here, and vice versa. Some particularly relevant embodiments may be pointed out or explicitly repeated.

It is appreciated that the expression “one or more exogenous nucleotide sequences including two or more genes” means that there may be one exogenous sequence which includes two or more genes, or there may be more than one exogenous sequence when all exogenous sequences together include two or more genes. When there is more than one exogenous nucleotide sequence, then each one of the exogenous sequences may include no genes, one gene, or more than one gene, as long as together they include two or more genes.

In some embodiments, the recombinant nucleic acid molecules are extrachromosomal. In some embodiments, the recombinant nucleic acid molecules are integrated into a plant chromosome.

In some embodiments, the plant is a pasture plant or a fodder plant. In some embodiments, the plant is a pasture plant. In some embodiments, the plant is a fodder plant.

The term “pasture plant” or “forage plant” is used herein to refer to plants which are planted or seeded in grazing fields for animals to graze on.

Nonlimiting examples for pasture plants which are grass include Agrostis spp. (bentgrasses), Agrostis capillaris (common bentgrass), Agrostis stolonifera (creeping bentgrass), Andropogon hallii (sand bluestem), Arrhenatherum elatius (false oat-grass), Bothriochloa bladhii (Australian bluestem), Bothriochloa pertusa (hurricane grass), Brachiaria spp. (signalgrass), Brachiaria decumbens (Surinam grass), Brachiaria humidicola (koronivia grass), Brachiaria ruziziensis, or any Brachiaria hybrids, Bromus spp. (bromegrasses), Cenchrus ciliaris (buffelgrass), Chloris gayana (Rhodes grass), Cynodon dactylon (bermudagrass), Dactylis glomerata (orchard grass), Echinochloa pyramidalis (antelope grass), Entolasia imbricata (bungoma grass), Festuca spp. (fescues), Festuca arundinacea (tall fescue), Festuca pratensis (meadow fescue), Festuca rubra (red fescue), Heteropogon contortus (black spear grass), Hymenachne amplexicaulis (West Indian marsh grass), Hyparrhenia rufa (jaragua), Leersia hexandra (southern cutgrass), Lolium spp. (ryegrasses), Lolium multiflorum (Italian ryegrass), Lolium perenne (perennial ryegrass), Megathyrsus maximus (Guinea grass), Melinis minutiflora (molasses grass), Paspalum conjugatum (carabao grass), Paspalum dilatatum (dallisgrass), Phalaris arundinacea (reed canarygrass), Phleum pratense (timothy), Poa spp. (bluegrasses, meadow-grasses), Poa arachnifera (Texas bluegrass), Poa pratensis (Kentucky bluegrass), Poa trivialis (rough bluegrass), Setaria sphacelata (African bristlegrass), Themeda triandra (kangaroo grass), and Thinopyrum intermedium (intermediate wheatgrass).

Nonlimiting examples for pasture plants which are legumes include Arachis pintoi (pinto peanut), Astragalus cicer (cicer milkvetch), Chamaecrista rotundifolia (roundleaf sensitive pea), Clitoria ternatea (butterfly-pea), Kummerowia (annual lespedezas), Kummerowia stipulacea (Korean clover, Korean lespedeza), Kummerowia striata (Japanese clover, common lespedeza), Lotus corniculatus (bird's-foot trefoil), Macroptilium atropurpureum (purple bush-bean), Macroptilium bracteatum (burgundy bean), Medicago spp. (medics), Medicago sativa (alfalfa, lucerne), Medicago truncatula (barrel medic), Melilotus spp. (sweetclovers), Neonotonia wightii (perennial soybean), Onobrychis viciifolia (common sainfoin), Stylosanthes spp. (stylo), Stylosanthes humilis (Townsville stylo), Stylosanthes scabra (shrubby stylo), Trifolium spp. (clovers), Trifolium hybridum (alsike clover), Trifolium incarnatum (crimson clover), Trifolium pratense (red clover), Trifolium repens (white clover), Vicia spp. (vetches), Vicia articulata (oneflower vetch), Vicia ervilia (bitter vetch), Vicia narbonensis (narbon vetch), Vicia sativa (common vetch, tare), Vicia villosa (hairy vetch), and Vigna parkeri (creeping vigna).

The term “fodder plant” is used herein to refer to plants which can be used to prepare livestock feedstuff. Nonlimiting examples for fodder plants include alfalfa, barley, duckweed, birdsfoot trefoil, Brassica (such as kale, rapeseed, rutabaga, and turnip), clover, grass, such as Bermuda grass, false oat grass, fescue, ryegrass, corn (maize), millet, oats, sorghum, soybeans, and wheat.

The terms “pasture plant” and “fodder plant” are not mutually exclusive, as many plants are part of both definitions.

In some embodiments, the plant is selected from a grass, a cereal, and a legume.

Examples for a grass include signalgrass (Brachiaria), ryegrass, tall fescue, cocksfoot, and kikuyu; examples for a cereal include corn, oats, and millet; and examples for a legume include alfalfa (Medicago sativa, lucerne), clover, and soy.

In some embodiments, the plant is selected from signalgrass (Brachiaria), alfalfa (Medicago sativa, lucerne), ryegrass, tall fescue, cocksfoot, clover, oats, millet, kikuyu, chicory, corn, soy, and plantain.

In some embodiments, the plant part is selected from a seed, a cell, a leaf, a stem, a root, a tuber, a cutting, a flower, a bark, a fruit, a bulb, a trichome, a rhizome, and any suitable part of a plant.

In some embodiments, the plant part is selected from a seed or a cell. In some embodiments, the plant part is an explant derived from any plant tissue.

In some embodiments, at least two of the two or more genes are from a species different from the plant species. In some embodiments, at least one of the two or more genes is from an organism selected from Asparagopsis taxiformis, Asparagopsis armata, Chondrus Crispus, Macrocystis pyrifera, Medicago sativa, and Medicago truncatula. In some embodiments, at least 2, 3, 4, 5, 6, or 7 of the two or more genes are from at least one organism selected from Asparagopsis taxiformis, Asparagopsis armata, Chondrus Crispus, Macrocystis pyrifera, Medicago sativa, and Medicago truncatula.

In some embodiments, at least one of the two or more genes is from a non-plant organism. In some embodiments, at least 2, 3, 4, or 5 of the two or more genes are from at least one non-plant organism. In some embodiments, the non-plant organism is an alga. In some embodiments, the alga is a red or brown alga. In some embodiments, the alga is a red alga. In some embodiments, the alga is selected from Asparagopsis taxiformis, Asparagopsis armata, Chondrus Crispus, and Macrocystis pyrifera. In some embodiments, the alga is Asparagopsis taxiformis.

In some embodiments, the genetically engineered plant or plant part is directly obtained by transformation with the system of the invention.

In some embodiments, the plant or plant part including the system of the invention is not directly obtained by transformation with the system of the invention but is derived from a plant or plant part which has been transformed with the system of the invention, or from a descendant of the transformed plant.

An additional way to reduce the amount of bromoform, further to the discussed above, is to dilute or mix the genetically engineered plants or plant parts with plants or plant parts which are not capable of producing bromoform, such as plants or plant parts not including the system of the invention. For example, genetically engineered seeds of the invention may be mixed with non-genetically engineered seeds to provide the amount of bromoform required for the ruminant animal.

Accordingly, in some embodiments, there is provided a composition including a mixture of the genetically engineered plants or plant parts of the invention (such as, e.g., seeds) with plants or plant parts not capable of producing bromoform. In some embodiments, the composition includes only plant parts, such as seeds, and no additional ingredients.

In some embodiments, up to about 5%, 10%, 15%, 20%, 25%, or 30% w/w, of the composition are genetically engineered plants or plant parts of the invention.

### Methods for Reducing Methane Emissions

The plants or plant parts of the invention, including the bromoform-producing genes described herein, are intended to be consumed by ruminant animals, such as cows. Two general directions exist to this end: preparation of livestock feedstuff based on the transformed plants, or seeding transformed pasture plants in grazing fields, to be consumed by ruminant animals.

Accordingly, in some embodiments, there is provided a method for reducing methane gas emission by a ruminant animal, the method including:
producing a genetically engineered seed of a pasture plant, the seed including two or more genes involved in bromoform production;
seeding the genetically engineered seed in a field where a ruminant animal grazes; and
growing a bromoform-producing pasture plant from the seed,
thereby causing the bromoform-producing pasture plant to be consumed by the ruminant animal.

In some embodiments, the method further includes a step of adding a source of bromine to the bromoform-producing pasture plant. In some embodiments, the method does not include adding a source of bromine to the bromoform-producing pasture plant.

A suitable source of bromine may include bromine-containing solutions when the bromine is in any suitable form, such as bromine or bromide, for example, a calcium bromide solution. The bromine (e.g. bromide) concentration in the bromine-containing solution may be about 1-100, 5-100, or 5-50 ppm. The source of bromine may be added by any suitable way, including spraying, irrigating, or dipping plant parts (such as plant leaves or roots) in a bromine-containing solution.

In some embodiments, the genetically engineered seed is produced by the method for preparing a genetically engineered plant or plant part disclosed herein.

In some embodiments, the bromoform-producing plants include the system disclosed herein.

Ruminants (suborder Ruminantia) or ruminant (ruminating) animals are hoofed herbivorous grazing or browsing mammals that acquire nutrients from plant-based food by fermenting it in a specialized stomach prior to digestion, principally through microbial actions. The process, which takes place in the front part of the digestive system and therefore is called foregut fermentation, typically requires the fermented ingesta (known as cud) to be regurgitated and chewed again. The process of rechewing the cud to further break down plant matter and stimulate digestion is called rumination. Digestion of food in the rumen (the first chamber and the primary site of microbial fermentation) is primarily carried out by the rumen microflora, which contains dense populations of several species of bacteria, protozoa, sometimes yeasts and other fungi. The hydrolysis of cellulose results in sugars, which are further fermented to acetate, lactate, propionate, butyrate, carbon dioxide, and methane. Methane is produced by a type of archaea, called methanogens, within the rumen, and this methane is released to the atmosphere. The rumen is the major site of methane production in ruminants.

In some embodiments, the ruminating animal is selected from cattle, a domesticated or a wild bovine (such as a cow), a goat, a sheep, a giraffe, a deer, a gazelle, and an antelope.

In some embodiments, the ruminant animal is a cow.

In some embodiments, there is provided a method for preparing a foodstuff for reducing methane gas emission by a ruminant animal, the method including:
producing a genetically engineered seed of a fodder plant, the seed including two or more genes involved in bromoform production;
seeding the genetically engineered seed;
growing a bromoform-producing fodder plant from the seed; and
preparing a foodstuff containing the bromoform-producing fodder plant or a part thereof.

Definitions and embodiments mentioned above and which may be relevant to the method embodiments also apply here, and vice versa. Some particularly relevant embodiments may be pointed out or explicitly repeated.

In some embodiments, the genetically engineered seed is produced by the method for preparing a genetically engineered plant or plant part disclosed herein.

In some embodiments, the bromoform-producing plants include the system disclosed herein.

In some embodiments, the ruminant animal is a cow.

It is noted that preparing animal foodstuff from plants is known in the art. The preparation may be conducted by wet or dry processing methods, including methods such as grinding, dry rolling, flaking, popping, micronizing, pressure cooking, roasting, extruding, pelleting, etc. Various nutritional ingredients may be added to the plant raw material.

In some embodiments, the ruminating animal is selected from cattle, a domesticated or a wild bovine (such as a cow), a goat, a sheep, a giraffe, a deer, a gazelle, and an antelope.

Unless defined otherwise, all technical and scientific terms used herein have the same meaning as commonly understood by one of ordinary skill in the art to which the invention pertains.

The term “a” and “an” refers to one or to more than one (i.e., to at least one, or to one or more) of the grammatical object of the article. By way of example, “an element” means one element or more than one element.

The term “about” when referring to a measurable value such as an amount, a ratio, and the like, is meant to encompass variations of ±10% of the indicated value, as such variations are also suitable to perform the disclosed invention. Any numerical values appearing in the application are intended to be construed as if preceded by “about”, unless indicated otherwise.

The term “nucleotide sequence” or “nucleic acid sequence”, as used herein, relates to a sequence of nucleotides and may include different types of nucleotides, such as DNA nucleotides, RNA nucleotides, and synthetic nucleotides.

The term “nucleic acid molecule” is a molecule including at least one nucleotide sequence. A nucleic acid molecule may be linear, circular, or branched, and the nucleotides may be modified or unmodified. In some embodiments, a nucleic acid molecule is a nucleic acid vector (usually a DNA vector) which includes elements such as genes, promoters, linkers, etc.

The term “recombinant”, as used herein with reference to a nucleic acid molecule or sequence, relates to a nucleic acid molecule or sequence that cannot be directly isolated from a natural organism, and which includes a combination of nucleotide sequences that are not present together in the same molecule in a natural organism.

The term “gene”, as used herein, relates to part of a nucleic acid sequence which is transcribed into RNA and optionally translated to a protein. Typically, a gene is operably linked to a promoter which drives transcription, and possibly to additional regulatory elements. In some embodiments, more than one gene is operably linked to a single promoter, such as in a prokaryotic operon. A gene may or may not include introns.

The term “bp”, as used herein, means base pair, or base pairs.

The term “aa”, as used herein, means amino acid or amino acids.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims which follow.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

The invention will now be illustrated by the following nonlimiting Examples.

### EXAMPLES

TABLE 1
Protein sequences

| Protein | Seq | |
|---|---|---|
| Name | ID | Protein sequence |
| Mbb1 | 1 | mtdtqnpnraevafkvrvsaaelarargspahlsnnsesrfrnpdgtrsllanftkglphiketalvesaidy |
| | | drfvraidsgdprdfadlplgpqgveprftsgiasdpevgtrawesggaglvfdlegpdaqavtmppape |
| | | ldsdelvaevtecywmsllrdvpfptfesnshiqaaaesinntqwikfknnppahltaaersrlrgpvttan |
| | | vfrgitpgdevgpylsqfllvgttgiangnevgdgfiqyggmrmdqrvrvakphidymttfgayldvqn |
| | | aanvsgrelykeeeprfrfihtprdlatyvhfdalyqaylnaciilldigapfdsgipfqldndidkqqgfatf |
| | | ggphilslvtevatralkavrfqkfnvhrrlrpeaigarvdrycatkapefagaaklsealdkellqkvhdhn |
| | | kkqnllsdrgnprandfnpdgevsegnllmpmafpegspmhpaygaghatvagacvtvlkaffdggy |
| | | rlpfcyitdedgtglqaveidepltvdgelnkicsnisigrnwagvhyftdyiesirigeeiaigilqeqkltfs |
| | | enfsmtlnkfdgstiri |
| Mbb2 | 2 | makqkslftllsatleshlsthafqllaliiyalanalmfvwgahdefhhhtnannlrwyiciargagytlnln |
| | | talvillaarlfatylretplqhilpldksfpafhivvaytiaaavvihasfhlawlvaydmwetgmwgftm |
| | | saatgvvllvvfivmfisampkyrkkhfrifylihsvgallffgllvfhgmynrvpetykwiaapliiytidr |
| | | vlrrykistaeleltgehsslkgsdilelrvpkpfdyqagqyaevcvksinsewhpftiassphedsmcfyi |
| | | kalgdwttnlrdafearvendlyeplkvqirgpfgapaqhvsgycrvvlisggvgstpfaaickhlhhlnk |
| | | senhskaleshasskrmsqvqwrirdaisilfdvslddsrndeaanqqrrqqladmlnmspkgdvhnvs |
| | | fkrtqskrsddfdsvedskqmnfslcrhdskssfnlddcsdairyynrpsfqffslstrhvinlyeyrtrllafl |
| | | httrftfallltlvarivilcivsifnlghiglynshidatwviatnsilglilgvallttilleisfmrmrffyrvw |
| | | rcvdffiflpitflcnisnfaswnghaqpkflifldliivlpimllllchrmyrsvgsrnllddtaecrqgckcnkti |
| | | pdvdfvwttprdsddewlrnelyplatgtelrlhryvtrenmadledperfittanagrpqwdeifaqiaeq |
| | | tpshskigvffcgphpmgaavqksmrkvevmsnlrgsylrktesavlvddlilrdegevkllreygcnirf |
| | | vfreenfs |
| Mbb3 | 3 | mtpptrrqtalvkriaaadlaanrhhphhvrnadettyklpnhvcsyisaftkglphhldgrlvsphhfqlfi |
| | | havrtghvkdiaaiplgpphsssfrsgiakdaavrgwesmaagntfdlqgpdaqavtmppcprinshel |
| | | vtemtelyymallrdvpfthfnkhhlvqsavydmnhthwtksnrcclprperrrhrskfttdnifrgvtpg |
| | | ditgpyisqfllvgtkgladvheetegyvqygatridqrvrtvqpkidymttwaayldvqngadlrrhsay |
| | | aeppnyrfictprdlatyvhydalyqaylnaciilldinapydsglpftaddavdkqqgfatfgaphvltlvt |
| | | evatralkavrfqkyavhrrlrpeavgglleqyrryggghsdlayiirpirhladslssdlmsmvakengrq |
| | | nslirdnghprshdegsddthllsmayaegspmhpsygaghatvagacvtilkaffdhtfklpfayvsssd |
| | | grklktvklskkltvedelnklaanisigrswagvhyysdyvesirlgeevaigmlkeqkltysekftmtip |
| | | kfdgsviei |
| Mbb4 | 4 | maeerrqnaleiriqaaklakkrdhpthkangdedrypetligsftkglpheketgllsnpadfadfvraint |
| | | gaikdmrrlklgidedprfisgiaskgdkhpfadtrawesmaagltydlegpdaqavtmppapkldsdel |
| | | vteitesywmallrdvpfteferdgntaaaaasisrtrwvqyneqpsqrpstltdeeiarlrgpytkknvfrg |
| | | vtngenvgpylsqfllvgtkgigdaqqvsdgyvqyggmrmdqrvrvavpkrdymttwaswldvqna |
| | | gdlrgreiydddtpfrfittprdlatwvhfdalyqaylnaciilldikapfdphipfqadddvdkqqgfatfg |
| | | gphilslctevatralkavrfqkynlhrrlrpeaigglverfkktngdpkfapvkklvndldgdmlrrveqh |
| | | nceqnklsddgharredyspegessqsyllpmafpegspmhpsygaghatvagacvtvlkaffdheyel |
| | | dfcyvpttdgkrlekvninekltvegelnklcanisigrnwagvhyysdyfesikvgeeiaigilqeqklty |
| | | gedffmtlpkfdgekiri |
| FabH | 5 | missilqgarilatgsaipdvcinnehlskivetsnewivtrtgieerriltgqnesiidlaslaskralekismn |
| | | pleidliilatsspddlfgsagqlqakigaskavafdltaacsgfvlgivtatqfiqnksyknilvvgadalsk |
| | | wtdwsdrstcilfgdaagaaiiqacseeengvlgfqlntngnksnqlsipyqthsyhldnhkslnlfqgrfq |
| | | yitmngqevykfavskvptsiaqclnslniskdeinwlllhqankrilyavadrlgidyskliynldkygnt |
| | | saasiplaldealakkqikyndiiviagfgagltwgttvikwkc |
| Sfp | 6 | msstrykipwltmniycfgrnlttascclppvqlpthkeahfwyvlpqevkctnllnryfeilspcekenvl |
| | | rmrgeelkksallaralvrttlaryqtncqinptslkfrkndygkpevdwqytddrslpplhfnishtssliac |
| | | gvtvgspigidveekqrrlkndilafarryfsphevemlahivdpelrrqefiklwtlkeayvkalgrgfsas |
| | | pfntftirlrdhvvkggihvppdviskeaeitvepsgdlknlssnwhfvlleligshyaaiciekdntnadkg |
| | | sipvnliirktipfvedgcisgtdsaevigglskllvc |
| ACCase | 7 | masvgrgngylnsvlpsrhpatttevdeycnalggnkpihsilianngmaavkfirsvrswayetfgteka |
| | | illvamatpedmrinaehiriadqfvevpggtnnnnyanvqlileiaeithvdavwpgwghasenpelp |
| | | dalkakgivflgppaismaalgdkigssliaqaaevptlpwsgshvkippesdlitipdeiyraacvyttee |
| | | aiascqvvgypamikaswggggkgirkvhnddevralfkqvqgevpgspifimkvasqsrhlevqlic |
| | | dqhgnfaalhsrdcsvqrrhqkiieegpitvappetvkeleqaarrlaksvnyvgaatveylysmetgeyy |
| | | flelnprlqvehpvtewiaeinlpaaqvavgmgiplwqipeirrfygmehgggndgwkktsvlatpfdf |
| | | deaqstkpkghcvavrvtsedpddgftptggkvqelsfkskpnvwayfsvksgggihefsdsqfghvfa |
| | | fgesralaianmvlglkeiqirgeirtnvdytidllnasdyrdnkihtgwldsriamrvraerppwylsvvg |
| | | galykasassaalvsdyvgylekggippkhislvhsqvslsiegskytidmvrggpgsyklklngseieae |
| | | ihtlrdggllmqldgnshviyaeeeaagtrllidgrtcllqndddpskligetpckllrylvaddsqidadtpy |
| | | aevevmkmcmpllspasgiihfrmaegqamqageliakldlddgsavrkaepftgsfpilgpptaisgk |
| | | vhqkcaaslnaarmilagyehnidevvvksllncldspelpflqwqecfavlatrlpkdlrneleakykefe |
| | | iisssqtidfpakllkaileahlsscpenekgalerlvepltslvksyeggreshahkivqslfeeylsveelfsd |
| | | niqadvierlrlqykkdllkivdivlshqgvksknklilrlmdklvypnpaayrdqlirfsqlnhivyselal |
| | | kasqlleqtklselrssiarslselemftedgenidtpkrksaindrmedlvsaplavedalvglfdhsdhtlq |
| | | rrvvetyirrlyqpylvkdsirmqwhrsgliatwefleeyverkngvedktlvekhsekkwgvmvviks |
| | | lqflpaiisaalreatnnfhdplksgsgdssnhgnmmhiglvginnqmsllqdsgdedqaqeridklakil |
| | | reqeigsiihaagvgdisciiqrdegrapmrhsfhwsseklyyveeplllhlepplsiyleldklkcyeniry |
| | | tpsrdrqwhlytvvdtkpqpiqrmflrtlirqpttnegyssyqrldaetsrtqlamsytsrsifrslmgameel |
| | | elnshnttiksehahmylyiireqqiddlvpyskkinieagqeettveaileelaqeihssvgvrmhrlgvf |
| | | vweiklwitacgqangawrvivnnvtghtctvhiyremedatthkvvyssvtvkgplhgvpvnenyqp |
| | | lggidrkrlaarknsttycydfplafqtsleqswsiqqtgiqrandkdllkvtelkfsekagswgtslvpaerl |
| | | pglndvgmvawlmemctpkfpsgrtilvvsndvtfkagsfgpredaffravtdlacakkipliylaansg |
| | | arlgvaeevkacfkvgwseeskpehgfqyvyltpedyarigssvmahelklesgetrwvidtivgkedgl |
| | | gvenlsgsgaiagaysrayketftltyvtgrtvgigaylarlgmrciqrldqpiiltgfsalnkllgrevysshm |
| | | qlggpkimatngvvhltvsddlegvssilkwlsyvpshvggalpivkpldppereveylpenscdpraai |
| | | sgtldvngkwlggifdkdsfvetlegwartvvtgraklggipvgivavetqtvmqiipadpgqldsherv |
| | | vpqagqvwfpdsatktaqaildfnreelplfiianwrgfsggqrdlfegilqagstivenlrtykqpifvyip |
| | | mmgelrggawvvvdsrinsdhiemyaertakgnvlepegmieikfrtrellecmrrldqqlinlkeklse |
| | | aksnkdygaydslqqqirfrekqllplytqiatkfaelhdtslrmkakgvirevldwrksrsvfyqrlhrrig |
| | | ehslinivrdaagdqlsyvsamnllkewylnsdiakgredawlddeaffrwrddpanyedklkelrvqrl |
| | | llqltnigdsaldlgalpqglaallskleassrdkliselrkvlg |

TABLE 2
Primers

| | | | | |
|---|---|---|---|---|
| | Seq | | Seq | Size |
| Primer 1 | ID | Primer 2 | ID | (bp) |

Validation primers

| | | | | | |
|---|---|---|---|---|---|
| Mbb1 | gcctgaatgtgtgaattgctctcg | 8 | tgacgcacaatcccactatc | 9 | 643 |
| Mbb2 | cttgtccaaagggagaatatgctgc | 10 | gatccgcatcctttcgcttc | 11 | 611 |
| Mbb3 | ccctggaggtcgaaggtattacc | 12 | tgacgcacaatcccactatc | 13 | 478 |
| Mbb4 | actgtcaagctttggtgctgg | 14 | gatccgcatcctttcgcttc | 15 | 722 |
| FabH | cttgtaggatttgttctgtatgaactggg | 16 | agatcgaagacgattttctgggt | 17 | 642 |
| Sfp | cattcttacgaaacttaaggctagttggg | 18 | agatcgaagacgattttctgggt | 19 | 568 |
| ACCase | gccatatcttgtcaaagatagcatcagg | 20 | ctctgtatgatacagctaatatctcc | 21 | 450 |
| | | | aacacc | | |
| Kan | gaacaagatggattgcacgc | 36 | gaagaactcgtcaagaaggc | 37 | 786 |
| Spect | ctcttcttgctgttcatctttatg | 38 | ccttagtaagaagaatagcaagat | 39 | 333 |
| | | | c | | |
| Basta | ctacgactggacggccg | 40 | gggggcatatccgagcg | 41 | 187 |

Expression primers

| | | | | | |
|---|---|---|---|---|---|
| Mbb1 | atgactgatacacagaaccctaatagagcc | 22 | gcctgaatgtgtgaattgctctcg | 23 | 533 |
| Mbb2 | atggctaagcagaagtccctc | 24 | cttgtccaaagggagaatatgctg | 25 | 303 |
| | | | c | | |
| Mbb3 | atgacccctcccaccag | 26 | ccctggaggtcgaaggtattacc | 27 | 368 |
| Mbb4 | atggctgaagaacgtagacag | 28 | actgtcaagctttggtgctgg | 29 | 420 |
| FabH | atgatttcaagtattttgcaaggggcc | 30 | cttgtaggatttgttctgtatgaact | 31 | 340 |
| | | | ggg | | |
| Sfp | atgtccagtaccagatataagatcccttgg | 32 | cattcttacgaaacttaaggctagtt | 33 | 414 |
| | | | ggg | | |
| ACCase | gccatatcttgtcaaagatagcatcagg | 34 | gccatatcttgtcaaagatagcatc | 35 | 450 |
| | | | agg | | |

Kan: kanamycin; Spetc: Spectinomycin

### Example 1: Cloning of Algae Bromoform Producing Genes

The gene transcripts encoding mbb1-mbb4 (Asparagopsis taxiformis), FabH (Chondrus crispus), sfp (Medicago truncatula), and ACCase (Medicago sativa) were synthesized with the codon usage of Medicago sativa and amplified by PCR with the indicated primers. The amplified DNA was cloned into binary vectors having a selective marker (spectinomycin, kanamycin, or basta) and transformed into Agrobacterium host cells. The genes were screened by colony PCR performed on the transformed cells grown on rifampicin and kanamycin (agrobacterial and vector selectable antibiotics). Several clones were characterized by whole plasmid sequencing as having complete coding sequences.

### Example 2: In-Planta Screen of Plants Expressing Bromoform

The genes were grouped in two different combinations as follows:

Group 1 (2 Plasmids with 4 Genes)

$$Plasmid 1 : mmb 1 + m bb 2 ( selective marker — Spectinomycin ) \\ Plasmid 2 : mbb 3 + mbb 4 ( selective marker — Kanamycin )$$

Group 2 (3 Plasmids with 7 Genes)

$$Plasmid 3 : sfp + mmb 1 + m bb 2 ( selective marker — Spectinomycin ) \\ Plasmid 4 : FabH + mbb 3 + mbb 4 ( selective marker — Kanamycin )$$

### Plasmid 5: ACCase (Selective Marker-Basta)

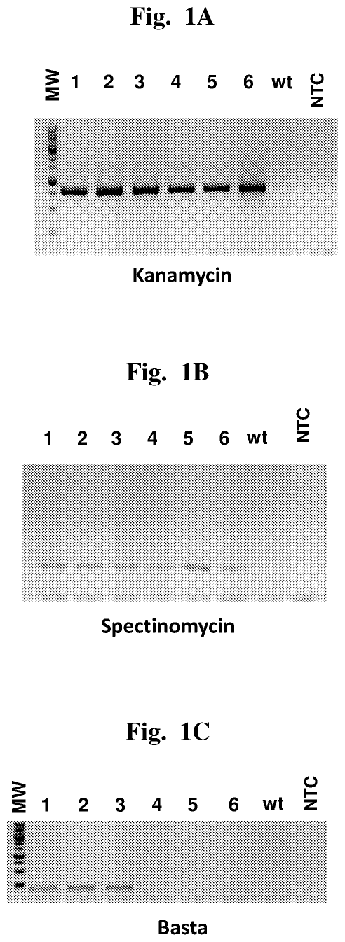

The different coding regions were cloned into pAB binary vectors under the control of the CaMV35S, act8, or ubq10 constitutive promoters and HSP, EFIA2, or GmPPO1 terminators, respectively, and different combinations of plasmids 1-5 (including each plasmid from 1-4 alone, and combinations including 1+2, 3+4, 1+2+5, and 3+4+5) were transformed into Arabidopsis plants using Agrobacterium according to the dipping flower method, essentially as described in WO 2018/178975. Briefly, plants were grown in soil composed of 75% peat, 25% perlite and irrigated routinely with water supplemented with fertilizer (e.g. Shefer 5.3.8, ICL Israel) according to manufacturer instructions, as needed. Plants started flowering after 3-4 weeks and were ready for transformation. Agrobacterium cultures transformed with the plasmids mentioned above were suspended in suspension buffer (5% sucrose and 0.03% L-77 Silwet) and flowers were dipped in the mixture for 1 minute. After 5-6 weeks of continued growth when the plants became dry, seeds were collected and kept in a cool dry place for 2 weeks or until used. Successful transformation of the plants is shown in FIG. 1.

TABLE 3
Gene structure

| Promoter | Gene | Terminator |
|---|---|---|
| act8 | Ms.mbb1 | HSP |
| CaMV35S | Ms.mbb2 | Gm |
| CaMV35S | Ms.mbb3 | HSP |
| act8 | Ms.mbb4 | GmPPO1 |
| ubq10 | Ms.FabH | EIFA2 |
| ubq10 | Ms.sfp | EIFA2 |
| CaMV35S | Ms.ACCase | HSP |

T1 generation transformed seeds were selected with a glufosinate ammonium selection (Basta, Bayer), kanamycin, spectinomycin or a combination of these selections, according to the manufacturer's instructions. Plants were irrigated with 10 ppm CaBr₂. Selected resistant plants are tested for bromoform production three-weeks post germination, as described in Example 7 below.

It is expected that plants capable of growing on the selectable markers, confirming successful transformation with bromoform-producing genes, will be able to produce bromoform.

### Example 3: Bromoform-Producing Tobacco Plants

Agrobacterium tumefaciens, GV3101 strain, was used to transform Tobacco plants (Nicotiana tabacum, cv. Little Dutch) with the above plasmids (combinations of plasmids 1-5, including 1+2, 3+4, 1+2+5, and 3+4+5). Each plasmid described above was transformed independently to the bacteria via electroporation. For the expression of several genes in the tobacco cells and plants, co-transformation with mixed bacteria were used. The Tobacco leaf explants were immersed in the A. tumefaciens suspension for 10 min, followed by placing in Petri dishes on solid co-cultivation medium (Gamborg's B5 basal salts with vitamins, 0.1 mM acetosyringone, 2 mg/ml BAP, 0.2 mg/ml NAA, 0.8% plant agar) and incubation at 25 C for 2-3 days in dark conditions. After cocultivation, explants were transferred to solidified shoot induction media (Gamborg's B5 basal salts with vitamins, 2 mg/L BAP, 0.2 mg/L NAA, 250 mg/L cefotaxime, selective antibiotics according to the plasmid; 100 mg/L kanamycin, 50 mg/L spectinomycin, 2 mg/L basta, 0.8% plant agar) and incubated at 25 C, 16 h light/8 h dark conditions. Every 14 days the explants were transferred to fresh media. Fully developed surviving shoots were planted on root induction medium (Gamborg's B5 basal salts with vitamins, 2 mg/L IBA, 250 mg/L cefotaxime, selective antibiotics according to the plasmid; 100 mg/L kanamycin, 50 mg/L spectinomycin, 2 mg/L basta, 0.8% plant agar) and grown until roots developed. After acclimation in propagators, the transgenic plants were transplanted to potting soil and maintained in a greenhouse. Successful transformation of the plants is shown in FIG. 1. Plants were irrigated with 10 ppm CaBr₂. Selected resistant three-weeks old plants are tested for bromoform production, as described in Example 7 below.

### Example 4: Testing Bromine Uptake by Wild-Type Tobacco Plants

Wild-type tobacco plants were used for testing bromine uptake through their root system from soil. Tobacco plants were grown in soil composed of 75% peat, 25% perlite and irrigated routinely with water supplemented with fertilizer (e.g. Shefer 5.3.8, ICL Israel) and watered regularly with water. From the age of one month, irrigation with CaBr₂ at a concentration of 5 ppm or 50 ppm was carried out twice a week for 3 consecutive weeks, and control plants continued to be watered without CaBr₂. 3 discs of 3 cm² were collected from each plant and used for bromine content analysis to evaluate bromine uptake.

TABLE 4
Bromine content analysis

| Extract | Treatment | Extract weight (mg) | Br (mg/kg) |
|---|---|---|---|
| 1 | control | 100.6 | 0.0 |
| 2 | control | 100.2 | 0.0 |
| 3 | 5 ppm | 100.3 | 19.9 |
| 4 | 5 ppm | 103 | 9.7 |
| 5 | 50 ppm | 100.3 | 10.0 |
| 6 | 50 ppm | 101.9 | 1265.9 |

The results presented in Table 4 indicate that plats can uptake bromine from the soil that can accumulate in their leaves. The bromine accumulation even to these high concentrations does not cause any detectable harm to the plants and to their respective growth and development.

### Example 5: Bromoform-Producing Alfalfa Plants

The above coding regions are cloned into pPA binary vectors as described above, and combinations of vectors 1-5, including 1+2, 3+4, 1+2+5, and 3+4+5 were used to transform alfalfa plants (Medicago sativa spp.) by Agrobacterium-mediated transformation. Seeds are collected from the transformants, planted, and screened for the presence of the construct.

Seeds are surface sterilized by submerging in 70% ethanol for 30 seconds. After the removal of ethanol, seeds are treated with 20% sodium hypochlorite and thoroughly washed with sterilized water 3 times. Individual seeds are then placed in the magenta boxes containing MS basal medium (Murashige and Skoog 1962), 30 g/L sucrose and 0.8% plant agar. Cotyledons and fully developed leaves from 2-3 week old plants are removed and cut into half on moist filter paper.

About 100 leaf and cotyledon explants are transferred to A. tumefaciens suspension (OD 0.5 at 600 nm) in infection medium (MS basal medium (Murashige and Skoog 1962), 30 g/L sucrose and 346 μM acetosyringone) in pH 5.8 and incubated for 20 minutes at room temperature. The explants are transferred to filter paper to prevent bacterial over-growth during co-culture access and transferred to co-cultivation media (Gamborg B5 medium (Gamborg 1984), 30 g/L sucrose, 2 mg/L 2,4-D, 0.1 mg/L kinetin and 346 μM acetosyringone) for 3 days in the dark.

Seven days after inoculation, the explants are washed with sterile water to remove the bacteria and blotted briefly on filter paper. 10-12 explants are transferred to callus induction medium (Gamborg B5 medium, 30 g/L sucrose, 0.8% plant agar, 2 mg/L 2,4-D, 0.1 mg/L kinetin, 500 mg/L cefotaxime sodium and selection reagent (according to the construct: 50 mg/L kanamycin/4 mg/L PPT/25 mg/L spectinomycin)). Explants are transferred to fresh media every 14 days until formation of somatic embryos. Dark green mature embryos are placed into plant development media (Gamborg B5 medium, 30 g/L sucrose, 0.8% plant agar, 0.1 mg/L myo-inositol, 500 mg/L cefotaxime sodium and selection reagent (according to the construct: 50 mg/L kanamycin/4 mg/L PPT/25 mg/L spectinomycin)). Calluses are transferred to fresh media every 14 days until shoots are formed. Green developed plantlets are transferred to rooting media (Gamborg B5 medium, 30 g/L sucrose, 0.8% plant agar, 0.1 mg/L myo-inosytol, 500 mg/L cefotaxime sodium). After acclimation in propagators, the transgenic plants are transplanted to potting soil and maintained in a greenhouse.

T1 generation transformed seeds are germinated on selection (as described above) according to the manufacturer's instructions. 3-4 days post germination, plants are irrigated with 5 ppm or 50 ppm CaBr₂. Selected resistant plants are tested for bromoform production three-weeks post germination.

It is expected that plants capable of growing on the selectable markers, confirming successful transformation with bromoform-producing genes, will also be able to produce bromoform.

### Example 6: Generation of a Genetically Engineered Brachiaria Plant

The above plasmids (combinations of plasmids 1-5, including 1+2, 3+4, 1+2+5, and 3+4+5) are transformed into Brachiaria (signalgrass) by Agrobacterium-mediated transformation, and seeds are collected from the transformants, planted, and screened for the presence of the construct.

Seeds are surface sterilized for 20 min in 20% sodium hypochlorite and thoroughly washed with sterilized water 3 times. Disinfected seeds are placed on solid agar medium containing 5 g/L sucrose and 6 g/L agar for 5 hours following soaking in distilled sterile water for overnight at room temperature in the dark. Embryos explants are isolated using a scalpel blade.

About 150 embryos are transferred to A. tumefaciens suspension (OD 0.5 at 600 nm) in infection medium (MS basal medium (Murashige and Skoog 1962), 30 g/L sucrose, 346 μM acetosyringone and 1 mM dithiothreitol) in pH 5.8 and sonicated for 30 seconds. Following sonication the explants are incubated for 2 hrs at room temperature and transferred to co-cultivation media (MS basal medium, 30 g/L sucrose, 100 mg/L casein hydrosylate, 2 mg/L 2,4-D, 0.2 mg/L BAP and 346 μM acetosyringone) with a single layer of autoclaved sterile filter paper for 3 days in the dark.

After co-cultivation, the base of each embryonic axis is embedded in callus induction medium (MS basal medium, 30 g/L sucrose, 0.8% plant agar, 300 mg/L casein hydrosylate, 2 mg/L 2,4-D, 0.2 mg/L BAP, 500 mg/L cefotaxime sodium and selection reagent (according to the construct: 50 mg/L kanamycin/4 mg/L PPT/25 mg/L spectinomycin)). The explants are transferred to fresh media every 14 days. Embryonic calluses are transferred to regeneration media (MS basal medium, 30 g/L sucrose, 0.8% plant agar, 300 mg/L casein hydrosylate, 100 mg/L myo-inosytol, 4 mg/L kinetin, 500 mg/L cefotaxime sodium and selection reagent (according to the construct: 50 mg/L kanamycin/4 mg/L PPT/25 mg/L spectinomycin)). Developed shoots are rooted in the presence of IAA mg/L and 15 g/L sucrose. After acclimation in propagators, the transgenic plants are transplanted to potting soil and maintained in a greenhouse.

It is expected that plants capable of growing on the selectable markers, confirming successful transformation with bromoform-producing genes, will also be able to produce bromoform.

### Example 7: Bromoform Production Testing

Leaves from transformed plants, optionally following irrigation with bromine (e.g. CaBr₂), as detailed above, are harvested, ground, and extracted. Extracts are assayed for their bromoform production capabilities by testing bromoform and its precursors by GC/LC-MS, as follows: ~500 mg of plant leaves are weighed into an HS-20 vial and the vial is cryogenically frozen in a beaker containing acetone and dry ice; the leaves are then ground until a fine powder is obtained, and 13 ml H₂O, 5.0 gr NaCl and 10 ul DCB (1,4-Dichlorobenzene) (~0.13 mg/ml) are added to the vial; the vial is tightly closed and inserted into a Headspace gas chromatography/mass spectrometry (GC/MS) analysis system.

1-37. (canceled)

38. A system for gene expression in a plant, comprising one or more recombinant nucleic acid molecules which comprise one or more nucleotide sequences together comprising two or more genes, wherein: each gene is operably linked to a promoter for expression in the plant; and the two or more genes encode at least one haloperoxidase enzyme and at least one other enzyme involved in bromoform production and not encoded by the plant.

39. The system of claim 38, wherein at least one of the two or more genes is from an organism selected from the group consisting of Asparagopsis taxiformis, Asparagopsis armata, Chondrus Crispus, Macrocystis pyrifera, Medicago sativa, and Medicago truncatula.

40. The system of claim 38, wherein at least two of the two or more genes are from a non-plant organism.

41. The system of claim 38, wherein the at least one other enzyme comprises an NAD(P)H Oxidase (NOX).

42. The system of claim 38, wherein the at least one other enzyme comprises at least one enzyme involved in fatty acid synthesis.

43. The system of claim 42, wherein the at least one enzyme involved in fatty acid synthesis is selected from the group consisting of Beta-ketoacyl-[acyl-carrier-protein]synthase III (FabH), phosphopantetheinyl transferase (sfp), acetyl-CoA carboxylase (ACCase), and malonyl CoA synthetase (MatB).

44. The system of claim 43, wherein the at least one other enzyme comprises NOX, and at least one enzyme involved in fatty acid synthesis selected from the group consisting of FabH, sfp, ACCase, and MatB.

45. The system of claim 44, wherein the at least one other enzyme comprises NOX, FabH, sfp, and ACCase.

46. The system of claim 38, wherein the two or more genes are selected from the group consisting of Mbb1 (A. taxiformis), Mbb2 (A. taxiformis), Mbb3 (A. taxiformis), Mbb4 (A. taxiformis), CcVHPO1 (Chondrus crispus), CcVHPO2 (Chondrus crispus), CcVHPO3 (Chondrus crispus), CcVHPO4 (Chondrus crispus), CcVHPO5 (Chondrus crispus), CcMbb2 (Chondrus crispus), Sfp, FaBH, ACCase, and MatB.

47. The system of claim 46, wherein the two or more genes comprise Mbb1, Mbb2, Mbb3, Mbb4, and at least one gene selected from the group consisting of FaBH, Sfp, ACCase, and MatB.

48. The system of claim 44, wherein: the haloperoxidase has at least 70, 75, 80, 85, 90, 95, or 99% sequence identity to SEQ ID NO: 1 (Mbb1), SEQ ID NO: 3 (Mbb3), or SEQ ID NO: 4 (Mbb4); the NOX has at least 70, 75, 80, 85, 90, 95, or 99% sequence identity to SEQ ID NO: 2 (Mbb2); the FabH has at least 70, 75, 80, 85, 90, 95, or 99% sequence identity to SEQ ID NO: 5; the sfp has at least 70, 75, 80, 85, 90, 95, or 99% sequence identity to SEQ ID NO: 6; and/or the ACCase has at least 70, 75, 80, 85, 90, 95, or 99% sequence identity to SEQ ID NO: 7.

49. The system of claim 38, wherein at least one of the promoters is a constitutive promoter.

50. The system of claim 38, wherein at least one of the promoters is an inducible promoter.

51. The system of claim 50, wherein the inducible promoter is a tissue-specific promoter, activated in plant organs which develop late in the plant life cycle, such as leaves or flowers.

52. The system of claim 38, wherein at least one of the two or more genes further encodes a chloroplast transit peptide for targeting the enzymes to a chloroplast.

53. The system of claim 38, wherein the plant is a pasture plant or a fodder plant.

54. The system of claim 38, wherein the plant is selected from the group consisting of signalgrass (Brachiaria), alfalfa (Medicago sativa, lucerne), ryegrass, tall fescue, cocksfoot, clover, oats, millet, kikuyu, chicory, corn, soy, and plantain.

55. A composition comprising the system of claim 38.

56. A genetically engineered bromoform-producing plant or plant part, comprising the system of claim 38.

57. The bromoform-producing plant or plant part of claim 56, wherein the plant part is selected from the group consisting of a seed, a cell, a leaf, a stem, a root, a tuber, a cutting, a flower, a bark, a fruit, a bulb, a trichome, and a rhizome.


SEQUENCE LISTING (41 sequences)

SEQ ID NO: 1 - protein, 590 aa, synthetic construct
MTDTQNPNRAEVAFKVRVSAAELARARGSPAHLSNNSESRFRNPDGTRSLLANFTKGLPHIKETALVESAIDYDRFVRAIDSGDPRDFADLPLGPQGVEPRFTSGIASDPEVGTRAWESGGAGLVFDLEGPDAQAVTMPPAPELDSDELVAEVTECYWMSLLRDVPFPTFESNSHIQAAAESINNTQWIKFKNNPPAHLTAAERSRLRGPVTTANVFRGITPGDEVGPYLSQFLLVGTTGIANGNEVGDGFIQYGGMRMDQRVRVAKPHIDYMTTFGAYLDVQNAANVSGRELYKEEEPRFRFIHTPRDLATYVHFDALYQAYLNACIILLDIGAPFDSGIPFQLDNDIDKQQGFATFGGPHILSLVTEVATRALKAVRFQKFNVHRRLRPEAIGARVDRYCATKAPEFAGAAKLSEALDKELLQKVHDHNKKQNLLSDRGNPRANDFNPDGEVSEGNLLMPMAFPEGSPMHPAYGAGHATVAGACVTVLKAFFDGGYRLPFCYITDEDGTGLQAVEIDEPLTVDGELNKICSNISIGRNWAGVHYFTDYIESIRIGEEIAIGILQEQKLTFSENFSMTLNKFDGSTIRI

SEQ ID NO: 2 - protein, 813 aa, synthetic construct
MAKQKSLFTLLSATLESHLSTHAFQLLALIIYALANALMFVWGAHDEFHHHTNANNLRWYICIARGAGYTLNLNTALVILLAARLFATYLRETPLQHILPLDKSFPAFHIVVAYTIAAAVVIHASFHLAWLVAYDMWETGMWGFTMSAATGVVLLVVFIVMFISAMPKYRKKHFRIFYLIHSVGALLFFGLLVFHGMYNRVPETYKWIAAPLIIYTIDRVLRRYKISTAELELTGEHSSLKGSDILELRVPKPFDYQAGQYAEVCVKSINSEWHPFTIASSPHEDSMCFYIKALGDWTTNLRDAFEARVENDLYEPLKVQIRGPFGAPAQHVSGYCRVVLISGGVGSTPFAAICKHLHHLNKSENHSKALESHASSKRMSQVQWRIRDAISILFDVSLDDSRNDEAANQQRRQQLADMLNMSPKGDVHNVSFKRTQSKRSDDFDSVEDSKQMNFSLCRHDSKSSFNLDDCSDAIRYYNRPSFQFFSLSTRHVINLYEYRTRLLAFLHTTRFTFALLLTLVARIVILCIVSIFNLGHIGLYNSHIDATWVIATNSILGLILGVALLTTILLEISFMRMRFFYRVWRCVDFFIFLPITFLCNISNFASWNGHAQPKFLIFLDLIIVLPIMLLLLCHRMYRSVGSRNLLDDTAECRQGCKCNKTIPDVDFVWTTPRDSDDEWLRNELYPLATGTELRLHRYVTRENMADLEDPERFITTANAGRPQWDEIFAQIAEQTPSHSKIGVFFCGPHPMGAAVQKSMRKVEVMSNLRGSYLRKTESAVLVDDLILRDEGEVKLLREYGCNIRFVFREENFS

SEQ ID NO: 3 - protein, 581 aa, synthetic construct
MTPPTRRQTALVKRIAAADLAANRHHPHHVRNADETTYKLPNHVCSYISAFTKGLPHHLDGRLVSPHHFQLFIHAVRTGHVKDIAAIPLGPPHSSSFRSGIAKDAAVRGWESMAAGNTFDLQGPDAQAVTMPPCPRLNSHELVTEMTELYYMALLRDVPFTHFNKHHLVQSAVYDMNHTHWTKSNRCCLPRPERRRHRSKFTTDNIFRGVTPGDITGPYISQFLLVGTKGLADVHEETEGYVQYGATRIDQRVRTVQPKIDYMTTWAAYLDVQNGADLRRHSAYAEPPNYRFICTPRDLATYVHYDALYQAYLNACIILLDINAPYDSGLPFTADDAVDKQQGFATFGAPHVLTLVTEVATRALKAVRFQKYAVHRRLRPEAVGGLLEQYRRYGGGHSDLAYIIRPIRHLADSLSSDLMSMVAKENGRQNSLIRDNGHPRSHDEGSDDTHLLSMAYAEGSPMHPSYGAGHATVAGACVTILKAFFDHTFKLPFAYVSSSDGRKLKTVKLSKKLTVEDELNKLAANISIGRSWAGVHYYSDYVESIRLGEEVAIGMLKEQKLTYSEKFTMTIPKFDGSVIEI

SEQ ID NO: 4 - protein, 586 aa, synthetic construct
MAEERRQNALEIRIQAAKLAKKRDHPTHKANGDEDRYPETLIGSFTKGLPHEKETGLLSNPADFADFVRAINTGAIKDMRRLKLGIDEDPRFISGIASKGDKHPFADTRAWESMAAGLTYDLEGPDAQAVTMPPAPKLDSDELVTEITESYWMALLRDVPFTEFERDGNTAAAAASISRTRWVQYNEQPSQRPSTLTDEEIARLRGPYTKKNVFRGVTNGENVGPYLSQFLLVGTKGIGDAQQVSDGYVQYGGMRMDQRVRVAVPKRDYMTTWASWLDVQNAGDLRGREIYDDDTPFRFITTPRDLATWVHFDALYQAYLNACIILLDIKAPFDPHIPFQADDDVDKQQGFATFGGPHILSLCTEVATRALKAVRFQKYNLHRRLRPEAIGGLVERFKKTNGDPKFAPVKKLVNDLDGDMLRRVEQHNCEQNKLSDDGHARREDYSPEGESSQSYLLPMAFPEGSPMHPSYGAGHATVAGACVTVLKAFFDHEYELDFCYVPTTDGKRLEKVNINEKLTVEGELNKLCANISIGRNWAGVHYYSDYFESIKVGEEIAIGILQEQKLTYGEDFFMTLPKFDGEKIRI

SEQ ID NO: 5 - protein, 338 aa, synthetic construct
MISSILQGARILATGSAIPDVCINNEHLSKIVETSNEWIVTRTGIEERRILTGQNESIIDLASLASKRALEKISMNPLEIDLIILATSSPDDLFGSAGQLQAKIGASKAVAFDLTAACSGFVLGIVTATQFIQNKSYKNILVVGADALSKWTDWSDRSTCILFGDAAGAAIIQACSEEENGVLGFQLNTNGNKSNQLSIPYQTHSYHLDNHKSLNLFQGRFQYITMNGQEVYKFAVSKVPTSIAQCLNSLNISKDEINWLLLHQANKRILYAVADRLGIDYSKLIYNLDKYGNTSAASIPLALDEALAKKQIKYNDIIVIAGFGAGLTWGTTVIKWKC

SEQ ID NO: 6 - protein, 330 aa, synthetic construct
MSSTRYKIPWLTMNIYCFGRNLTTASCCLPPVQLPTHKEAHFWYVLPQEVKCTNLLNRYFEILSPCEKENVLRMRGEELKKSALLARALVRTTLARYQTNCQINPTSLKFRKNDYGKPEVDWQYTDDRSLPPLHFNISHTSSLIACGVTVGSPIGIDVEEKQRRLKNDILAFARRYFSPHEVEMLAHIVDPELRRQEFIKLWTLKEAYVKALGRGFSASPFNTFTIRLRDHVVKGGIHVPPDVISKEAEITVEPSGDLKNLSSNWHFVLLELIGSHYAAICIEKDNTNADKGSIPVNLIIRKTIPFVEDGCISGTDSAEVIGGLSKLLVC

SEQ ID NO: 7 - protein, 2257 aa, synthetic construct
MASVGRGNGYLNSVLPSRHPATTTEVDEYCNALGGNKPIHSILIANNGMAAVKFIRSVRSWAYETFGTEKAILLVAMATPEDMRINAEHIRIADQFVEVPGGTNNNNYANVQLILEIAEITHVDAVWPGWGHASENPELPDALKAKGIVFLGPPAISMAALGDKIGSSLIAQAAEVPTLPWSGSHVKIPPESDLITIPDEIYRAACVYTTEEAIASCQVVGYPAMIKASWGGGGKGIRKVHNDDEVRALFKQVQGEVPGSPIFIMKVASQSRHLEVQLICDQHGNFAALHSRDCSVQRRHQKIIEEGPITVAPPETVKELEQAARRLAKSVNYVGAATVEYLYSMETGEYYFLELNPRLQVEHPVTEWIAEINLPAAQVAVGMGIPLWQIPEIRRFYGMEHGGGNDGWKKTSVLATPFDFDEAQSTKPKGHCVAVRVTSEDPDDGFTPTGGKVQELSFKSKPNVWAYFSVKSGGGIHEFSDSQFGHVFAFGESRALAIANMVLGLKEIQIRGEIRTNVDYTIDLLNASDYRDNKIHTGWLDSRIAMRVRAERPPWYLSVVGGALYKASASSAALVSDYVGYLEKGQIPPKHISLVHSQVSLSIEGSKYTIDMVRGGPGSYKLKLNQSEIEAEIHTLRDGGLLMQLDGNSHVIYAEEEAAGTRLLIDGRTCLLQNDDDPSKLIGETPCKLLRYLVADDSQIDADTPYAEVEVMKMCMPLLSPASGIIHFRMAEGQAMQAGELIAKLDLDDGSAVRKAEPFTGSFPILGPPTAISGKVHQKCAASLNAARMILAGYEHNIDEVVVKSLLNCLDSPELPFLQWQECFAVLATRLPKDLRNELEAKYKEFEIISSSQTIDFPAKLLKAILEAHLSSCPENEKGALERLVEPLTSLVKSYEGGRESHAHKIVQSLFEEYLSVEELFSDNIQADVIERLRLQYKKDLLKIVDIVLSHQGVKSKNKLILRLMDKLVYPNPAAYRDQLIRFSQLNHIVYSELALKASQLLEQTKLSELRSSIARSLSELEMFTEDGENIDTPKRKSAINDRMEDLVSAPLAVEDALVGLFDHSDHTLQRRVVETYIRRLYQPYLVKDSIRMQWHRSGLIATWEFLEEYVERKNGVEDKTLVEKHSEKKWGVMVVIKSLQFLPAIISAALREATNNFHDPLKSGSGDSSNHGNMMHIGLVGINNQMSLLQDSGDEDQAQERIDKLAKILREQEIGSIIHAAGVGDISCIIQRDEGRAPMRHSFHWSSEKLYYVEEPLLLHLEPPLSIYLELDKLKCYENIRYTPSRDRQWHLYTVVDTKPQPIQRMFLRTLIRQPTTNEGYSSYQRLDAETSRTQLAMSYTSRSIFRSLMGAMEELELNSHNTTIKSEHAHMYLYIIREQQIDDLVPYSKKINIEAGQEETTVEAILEELAQEIHSSVGVRMHRLGVFVWEIKLWITACGQANGAWRVIVNNVTGHTCTVHIYREMEDATTHKVVYSSVTVKGPLHGVPVNENYQPLGGIDRKRLAARKNSTTYCYDFPLAFQTSLEQSWSIQQTGIQRANDKDLLKVTELKFSEKAGSWGTSLVPAERLPGLNDVGMVAWLMEMCTPKFPSGRTILVVSNDVTFKAGSFGPREDAFFRAVTDLACAKKIPLIYLAANSGARLGVAEEVKACFKVGWSEESKPEHGFQYVYLTPEDYARIGSSVMAHELKLESGETRWVIDTIVGKEDGLGVENLSGSGAIAGAYSRAYKETFTLTYVTGRTVGIGAYLARLGMRCIQRLDQPIILTGFSALNKLLGREVYSSHMQLGGPKIMATNGVVHLTVSDDLEGVSSILKWLSYVPSHVGGALPIVKPLDPPEREVEYLPENSCDPRAAISGTLDVNGKWLGGIFDKDSFVETLEGWARTVVTGRAKLGGIPVGIVAVETQTVMQIIPADPGQLDSHERVVPQAGQVWFPDSATKTAQAILDFNREELPLFIIANWRGFSGGQRDLFEGILQAGSTIVENLRTYKQPIFVYIPMMGELRGGAWVVVDSRINSDHIEMYAERTAKGNVLEPEGMIEIKFRTRELLECMRRLDQQLINLKEKLSEAKSNKDYGAYDSLQQQIRFREKQLLPLYTQIATKFAELHDTSLRMKAKGVIREVLDWRKSRSVFYQRLHRRIGEHSLINIVRDAAGDQLSYVSAMNLLKEWYLNSDIAKGREDAWLDDEAFFRWRDDPANYEDKLKELRVQRLLLQLTNIGDSALDLQALPQGLAALLSKLEASSRDKLISELRKVLG

SEQ ID NO: 8 - DNA, 24 bp, synthetic construct
  source 1..24: mol_type = other DNA
gcctgaatgtgtgaattgctctcg

SEQ ID NO: 9 - DNA, 20 bp, synthetic construct
  source 1..20: mol_type = other DNA
tgacgcacaatcccactatc

SEQ ID NO: 10 - DNA, 25 bp, synthetic construct
  source 1..25: mol_type = other DNA
cttgtccaaagggagaatatgctgc

SEQ ID NO: 11 - DNA, 20 bp, synthetic construct
  source 1..20: mol_type = other DNA
gatccgcatcctttcgcttc

SEQ ID NO: 12 - DNA, 23 bp, synthetic construct
  source 1..23: mol_type = other DNA
ccctggaggtcgaaggtattacc

SEQ ID NO: 13 - DNA, 20 bp, synthetic construct
  source 1..20: mol_type = other DNA
tgacgcacaatcccactatc

SEQ ID NO: 14 - DNA, 21 bp, synthetic construct
  source 1..21: mol_type = other DNA
actgtcaagctttggtgctgg

SEQ ID NO: 15 - DNA, 20 bp, synthetic construct
  source 1..20: mol_type = other DNA
gatccgcatcctttcgcttc

SEQ ID NO: 16 - DNA, 29 bp, synthetic construct
  source 1..29: mol_type = other DNA
cttgtaggatttgttctgtatgaactggg

SEQ ID NO: 17 - DNA, 23 bp, synthetic construct
  source 1..23: mol_type = other DNA
agatcgaagacgattttctgggt

SEQ ID NO: 18 - DNA, 29 bp, synthetic construct
  source 1..29: mol_type = other DNA
cattcttacgaaacttaaggctagttggg

SEQ ID NO: 19 - DNA, 23 bp, synthetic construct
  source 1..23: mol_type = other DNA
agatcgaagacgattttctgggt

SEQ ID NO: 20 - DNA, 28 bp, synthetic construct
  source 1..28: mol_type = other DNA
gccatatcttgtcaaagatagcatcagg

SEQ ID NO: 21 - DNA, 32 bp, synthetic construct
  source 1..32: mol_type = other DNA
ctctgtatgatacagctaatatctccaacacc

SEQ ID NO: 22 - DNA, 30 bp, synthetic construct
  source 1..30: mol_type = other DNA
atgactgatacacagaaccctaatagagcc

SEQ ID NO: 23 - DNA, 24 bp, synthetic construct
  source 1..24: mol_type = other DNA
gcctgaatgtgtgaattgctctcg

SEQ ID NO: 24 - DNA, 21 bp, synthetic construct
  source 1..21: mol_type = other DNA
atggctaagcagaagtccctc

SEQ ID NO: 25 - DNA, 25 bp, synthetic construct
  source 1..25: mol_type = other DNA
cttgtccaaagggagaatatgctgc

SEQ ID NO: 26 - DNA, 17 bp, synthetic construct
  source 1..17: mol_type = other DNA
atgacccctcccaccag

SEQ ID NO: 27 - DNA, 23 bp, synthetic construct
  source 1..23: mol_type = other DNA
ccctggaggtcgaaggtattacc

SEQ ID NO: 28 - DNA, 21 bp, synthetic construct
  source 1..21: mol_type = other DNA
atggctgaagaacgtagacag

SEQ ID NO: 29 - DNA, 21 bp, synthetic construct
  source 1..21: mol_type = other DNA
actgtcaagctttggtgctgg

SEQ ID NO: 30 - DNA, 27 bp, synthetic construct
  source 1..27: mol_type = other DNA
atgatttcaagtattttgcaaggggcc

SEQ ID NO: 31 - DNA, 29 bp, synthetic construct
  source 1..29: mol_type = other DNA
cttgtaggatttgttctgtatgaactggg

SEQ ID NO: 32 - DNA, 30 bp, synthetic construct
  source 1..30: mol_type = other DNA
atgtccagtaccagatataagatcccttgg

SEQ ID NO: 33 - DNA, 29 bp, synthetic construct
  source 1..29: mol_type = other DNA
cattcttacgaaacttaaggctagttggg

SEQ ID NO: 34 - DNA, 28 bp, synthetic construct
  source 1..28: mol_type = other DNA
gccatatcttgtcaaagatagcatcagg

SEQ ID NO: 35 - DNA, 28 bp, synthetic construct
  source 1..28: mol_type = other DNA
gccatatcttgtcaaagatagcatcagg

SEQ ID NO: 36 - DNA, 20 bp, synthetic construct
  source 1..20: mol_type = other DNA
gaacaagatggattgcacgc

SEQ ID NO: 37 - DNA, 20 bp, synthetic construct
  source 1..20: mol_type = other DNA
gaagaactcgtcaagaaggc

SEQ ID NO: 38 - DNA, 24 bp, synthetic construct
  source 1..24: mol_type = other DNA
ctcttcttgctgttcatctttatg

SEQ ID NO: 39 - DNA, 25 bp, synthetic construct
  source 1..25: mol_type = other DNA
ccttagtaagaagaatagcaagatc

SEQ ID NO: 40 - DNA, 17 bp, synthetic construct
  source 1..17: mol_type = other DNA
ctacgactggacggccg

SEQ ID NO: 41 - DNA, 17 bp, synthetic construct
  source 1..17: mol_type = other DNA
gggggcatatccgagcg